(19) United States
(12) Patent Application Publication
(10) Pub. No.: US 2010/0271595 A1
(43) Pub. Date: Oct. 28, 2010

(54) Device for and method of ray tracing wave front conjugated aberrometry

(76) Inventor: Vasyl Molebny, Kiev (UA)

Correspondence Address: Vasyl Molebny, 6 Velyka Kytaivska St., Apt. 9, Kiev, 03028, omitted

(21) Appl. No.: 12/428,474

(22) Filed: Apr. 23, 2009

(51) Int. Cl. A61B 3/10

(52) U.S. Cl. 351/221; 351/246

(57) ABSTRACT

Two stages of ray tracing aberrometry include preliminary stage of measurement with probing beams successively entering the eye in parallel to the optical axis and the main stage of measurement with probing beams successively entering the same points of the eye but tilted in the way to compensate for the refraction variations over the entrance aperture measured in the preliminary stage. The main stage of measurement may be implemented in the combination of units, one compensating for defocus another—compensating for higher order aberrations. In one embodiment, the probing channel contains two two-coordinate acousto-optic deflectors with a collimating lens between them. The procedure of main stage of measurement may be iteratively repeated until the wave front conjugation is achieved with a prescribed accuracy.

### FIELD OF THE INVENTION

The present invention relates to ophthalmic instruments that are used to examine the eye, in particular to ophthalmic examination instruments that measure and characterize the aberrations of the human eye, especially to those of them that provide high accuracy of measurements.

### BACKGROUND OF THE INVENTION

Early instruments for measurement of aberrations of the human eye, called also instruments for wave front measurement, used the feedback that could be subjective or objective (setting the feedback signal to zero). Examples of such systems were described by S. M. Smirnov (Measurement of the wave aberration of the human eye. Biophysics, 1961, No. 6, pp. 776-795) and C. M. Penney et al (U.S. Pat. No. 5,258,791).

The tendency to automate the measurements and make them faster resulted in several commercialized technologies. One of them is Hartmann-Shack wave front sensor whose principle was borrowed from the astronomy and military applications by J. Liang et al, (“Objective measurement of wave aberrations of the human eye with the use of a Hartmann-Shack wave-front sensor”. Journal of the Optical Society of America, 1994, Vol. 11, No. 7, pp. 1949-1957). According to this method, a point source produced on the retina of a living eye by a laser beam is reflected from the retina and received at a lenslet array of a Hartmann-Shack wavefront sensor such that each of the lenslets forms an image of the retinal point source on a CCD camera. From these data, wave front map is reconstructed, as well refraction error map.

Another, ray tracing approach was proposed by V. Molebny et al. (U.S. Pat. No. 6,932,475). According to this method, a point-by-point procedure is applied to probe the eye with a thin laser beam, to get the image of its projection on the retina, to measure the position of the trace of the laser beam on retina for each entrance point, and to reconstruct the wave front map, refraction error map, and other derivative characteristics from these data.

The principle of simultaneous projection of regular structure of light on the retina was implemented in the aberrometer described by P. Mierdel et al. (“Ocular optical aberrometer for clinical use”. Journal of Biomedical Optics. 2001, Vol. 6, No. 2, pp. 200-204). Its principle goes back to the Tscherning aberroscope. A collimated laser beam illuminates a mask with a regular matrix of holes which forms a bundle of thin parallel rays of 0.3 mm diameter. These rays are focused by a lens in front of the eye so that their intraocular focus point is located a certain distance in front of the retina, generating a corresponding pattern of light spots on it. The retinal spot pattern is imaged by a video camera. Deviations of all spots from their ideal regular positions are measured, and from these values the wave front aberration is computed.

Still another principle of aberration measurement was proposed by M. Fujieda (U.S. Pat. No. 5,907,388), its implementation in Nidek aberrometer being described by S MacRae et al. (“Slit skiascopic-guided ablation using the Nidek laser”. Journal of Refractive Surgery, 2000, Vol. 16, No. 5, pp. S576-S580). Moving strips of light are projected on the retina, their images are detected and the phases are measured, these phases being indicative of the degree of ametropia along the direction of the movement of strips.

Each of these commercially available aberrometers has its own limitations that can be overcome by specific measures like fast acousto-optic scanning in ray tracing, special information processing to resolve ambiguities in highly aberrated eyes when using Hartmann-Shack sensors, etc. Physiologically, the most correct is the ray tracing aberrometer since it uses the natural paths of light in the eye projecting the image of outer world on the retina. In the aberrometer using the Hartmann-Shack sensor, the path from the eye is not identical to that along which the optical system of the eye traces the image of the outer world. Therefore, the results with Shack-Hartmann are correct only when there are no aberrations. In Tscherning and skiascopic aberrometers, measuring light is projected on retinal area not corresponding to the area used for vision.

To achieve higher accuracy, several methods and devices were proposed to modify the devices for and methods of aberration measurement of the optical system of the human eye. Initially, J. Ling described an idea of achieving a supernormal vision, as a copy of the astronomy techniques (“Supernormal vision and high-resolution retinal imaging through adaptive optics”. Journal of the Optical Society of America, 1994, Vol. 14, No. 11, pp. 2884-2892). Applying this approach to measure the aberrations, D. R. Williams et al. (U.S. Pat. No. 5,777,719) used the output signal from the device for wave front measurement to control a wave front compensation device (a deformable mirror) making it to take an appropriate shape and provide wave front compensation for the aberrations of the eye.

B. M. Levine et al. (U.S. Pat. No. 6,709,108) described an ophthalmic instrument for in-vivo examination of a human eye including a wavefront sensor that estimates aberrations in reflections of the light formed as an image on the retina of the human eye and a phase compensator that spatially modulates the phase of incident light to compensate for the estimated aberrations. The compensated image is recreated at the human eye to provide the human eye with a view of compensation of its aberrations.

C. Campbell (U.S. Pat. No. 7,128,416) proposed a method for measuring an optical aberration of an optical system of the human eye that comprises an adaptive optic disposed along the optical path between the optical system of the eye and the sensor. The adaptive optic is adjusted in response to a signal generated by the aberration sensor so as to provide a desired sensed aberration to compensate for the wave front distortions, i.e., to provide the wave front conjugation.

Unfortunately, as noted C. Campbell (U.S. Pat. No. 7,128,416), the adjusted shape of the deformable mirror does not directly indicate to the physician the actual aberrations of the patient's eye. Consequently, it is often required to apply a complicated calibration scheme so that the control signals used to deform the deformable mirror may be correlated with the aberrations from the patient's eye that the deformed mirror removes.

Another shortcoming of wave front conjugation with active optics is the following: in general case, the coordinates of the elementary mirrors controlling the wave front tilt do not coincide with the coordinates of the eye aperture in which the wave front is measured. It means that the values of the necessary wave front tilt in the control points are not measured directly, but should be approximated from the data acquired in other points.

Still another drawback is in involvement of subjective perception in some of the above reviewed techniques to judge how perfect the conjugation is made.

Still another problem of wave front conjugation with active optics is its high cost, especially when using photolithographic high spatial density MEMS (Micro-Electro-Mechanical Structure) technologies (F.-Y. Chen et al., U.S. Pat. No. 7,205,176).

There is thus a need for, and it would be highly advantageous to have a device for and a method of objective wave front conjugated aberrometry capable of high accuracy of measurement due to wave front conjugation in the same points where the measurement is taken thus representing an actual value of the aberration to the user and being cheaper in their cost as compared to the high spatial density MEMS technologies.

It is therefore an object of the invention to provide improved device for and method of wave ray tracing front conjugated aberrometry in which the above mentioned shortcomings are essentially neutralized.

### SUMMARY OF THE INVENTION

According to the present invention there is provided a device for ray tracing wave front conjugated aberrometry, containing a positioning and accommodation channel, a probing channel, a detection channel, and an information processing and control channel, with the probing channel consisting of a laser, a first scanning unit, a collimating lens, and a telescope, said first scanning unit having sequentially installed a first x-deflecting acousto-optic crystal connected to a first x-driver, a telescope, and a first y-deflecting acousto-optic crystal connected to a first y-driver, in which a second scanning unit is installed between the collimating lens and the telescope of said probing channel consisting of a second x-deflecting acousto-optic crystal connected to a second x-driver, a telescope, and a second y-deflecting acousto-optic crystal connected to a second y-driver, both said second x-driver and said second y-driver are connected to said information processing and control channel.

According to another feature of the present invention there is provided a method for ray tracing wave front conjugated aberrometry based on successively projecting thin laser beams on retina through a set of points of the eye entrance aperture, measuring the coordinates of the projected laser spots on retina, calculating the wave front tilt in each entrance point from the known coordinates of the entrance points and measured coordinates of the projected laser spots on retina, reconstructing the wave front map using mathematical methods of interpolation or approximation and any other derivative characteristic comprising the conjugation of the laser beam tilt at the entrance into the eye to compensate for the tilt induced by the aberrations along the beam path in the eye in the steps of calculation of the beam tilt at the entrance into the eye in a point with known coordinates, back-tracing the beam to determine its coordinates at the exit of the second scanning unit, calculation of the angle of deflection in the first scanning unit, applying the voltages to the crystals of the first scanning unit with the frequencies corresponding to the calculated angles of deflection, applying the voltages to the crystals of the second scanning unit with the frequencies corresponding to the calculated angles of deflection, repeating the steps of consecutively projecting thin laser beams on retina through the initially designated set of points of eye entrance aperture at the angles as defined by the above prescribed procedures and reconstructing the wave front map and any other derivative characteristic using mathematical methods of interpolation or approximation.

According to further features in preferred embodiments of the invention described below, there is provided a method wherein the steps of measurement with corrected angles of deflection are repeated iteratively until the deviation of the laser spots on retina from a central position is less than specified in advance.

According to still further features in the preferred embodiments, there is provided a device wherein a defocus compensator consisting of two lenses forming a telescope and two reflecting surfaces between said two lenses is installed at the entrance of the eye on the path common for the probing channel and for the detecting channel, said reflecting surfaces being oriented at 45 degrees to the optical axis, the back focus of the last lens of said defocus compensator coincides with the nodal point of the eye.

According to still further features in the preferred embodiments, there is provided a method wherein the conjugation of the laser beam tilt at the entrance into the eye compensating for the tilt induced by the aberrations along the beam path in the eye is performed separately for defocus component of the eye aberrations—by the defocus compensator and for all the rest of aberration components—by the first and the second scanning units.

According to still further features in the preferred embodiments, there is provided a device wherein a magnifying telescope is installed at the exit of the second scanning unit, the front focus of its first lens coinciding with the center of scanning of the second scanning unit.

The above and other features and advantages of the present invention will become more apparent in the following drawings, detailed description, and claims.

### BRIEF DESCRIPTION OF THE DRAWINGS

FIG. 1 is an optical layout of the device for wave front conjugated ray tracing aberrometry with electronic and electro-mechanic elements illustrating a possible embodiment of the present invention.

FIG. 2 shows an example of a set of points within the pupil of the eye in which the laser beam is projected.

FIG. 3 is an example of a retina spot diagram as reproduced by the ray tracing aberrometer with the set of entrance points shown in FIG. 2.

FIG. 4 illustrates a decomposition of a reconstructed surface into Zernike polynomials, horizontal axis showing the index of Zernike coefficient, vertical axis showing the value of the coefficient in micrometers.

FIG. 5 is an example of a reconstructed wave front called a “Wavefront Map”. It is a two-dimensional surface corresponding to the decomposition illustrated in FIG. 4. The values of wave front deviation from the reference surface measured in micrometers are coded by colors.

FIG. 6 is an example of reconstructed refraction errors called a “Refraction Map”. It is a two-dimensional surface corresponding to the decomposition illustrated in FIG. 4. The values of refraction errors, i.e. deviations from the emmetropia, measured in diopters are coded by colors.

FIG. 7 illustrates one of the derivative characteristics—a “Point Spread Function” that is a distribution of light intensity on retina formed by the optical system of the eye as an image of a far point object.

FIG. 8 illustrates another derivative characteristic—a “Modulation Transfer Function” showing how the contrast of an image degrades in the optical system of the eye at different spatial frequencies. The contrast is measured in parts of a unit, spatial frequency—in cycles/degree.

FIG. 9 shows the ray traces in one of the device implementations, where the elements are depicted in their equivalents. The first scanning unit 18 is depicted as a single plane of the centers of scanning (centers of scanning in x and y directions are combined due to intermediate telescope 26-27). The second scanning unit 20 is also depicted as a single plane of the centers of scanning (centers of scanning in x and y directions are combined due to intermediate telescope 32-33). The collimating lens 19 is depicted as a thin lens. The eye 6 is represented by its simplest model. FIG. 9A corresponds to a hyperopic eye, FIG. 9B—to a myopic eye.

FIG. 10 is an example of the retina spot diagram acquired after a complete wave front conjugation.

FIG. 11 is an example of the retina spot diagram acquired after a non-complete wave front conjugation.

FIG. 12 illustrates the principle of compensation of the defocus component of eye aberrations by the defocus compensator 4 of FIG. 1, where FIG. 12A corresponds to an emmetropic eye (movable mirrors 42-43 are in the initial position), FIG. 12B—to a myopic eye (movable mirrors 42-43 are shifted to shorten the distance between the telescope lenses 40 and 41), FIG. 12C—to a hyperopic eye (movable mirrors 42-43 are shifted to make longer the distance between the telescope lenses 40 and 41).

FIG. 13 illustrates the result of compensation of the defocus component of eye aberrations showing a zero defocus component in Zernike decomposition.

FIG. 14 shows the ray traces (the case of a hyperopic eye) in another implementation of the present invention, where in addition to the elements depicted in FIG. 9 in their equivalents, the defocus compensator 4 (FIG. 1) is depicted in the thin-lens equivalents of the lenses 40 and 41. FIG. 14A corresponds to a zero-deflection position of the second scanning unit 20 (plane 30-31) and initial position of the defocus compensator 4 (lenses 40 and 41), FIG. 14B demonstrates the action of the defocus compensator 4 with the changed distance between lenses 40 and 41. FIG. 14C illustrates a complete compensation of the aberrations of the eye corresponding to a complete wave front conjugation.

FIG. 15 shows the ray traces (the case of a myopic eye) in the same implementation of the present invention as in FIG. 14. In addition to the elements depicted in FIG. 9 in their equivalents, the defocus compensator 4 (FIG. 1) is depicted in the thin-lens equivalents of the lenses 40 and 41. FIG. 15A corresponds to a zero-deflection position of the second scanning unit 20 (plane 30-31) and initial position of the defocus compensator 4 (lenses 40 and 41), FIG. 15B demonstrates the action of the defocus compensator 4 with the changed distance between lenses 40 and 41. FIG. 15C illustrates a complete compensation of the aberrations of the eye corresponding to a complete wave front conjugation.

FIG. 16 illustrates the effect of defocus compensation for the detection process. FIG. 16A corresponds to the emmetropic eye, FIG. 16B—to the myopic eye, FIG. 16C—to the hyperopic eye.

FIG. 17 demonstrates the shape of intensity distribution in the plane of the detector. FIG. 17A corresponds to the signal from the eye of the patient with 10 diopter non-compensated hyperopia.

FIG. 17B shows the signal after compensation of the ametropia using the defocus compensator 4.

### DETAILED DESCRIPTION OF THE INVENTION

The embodiments of a device for and a method of ray tracing wave front conjugated aberrometry according to the present invention will be described in detail hereinafter with reference to the accompanying drawings.

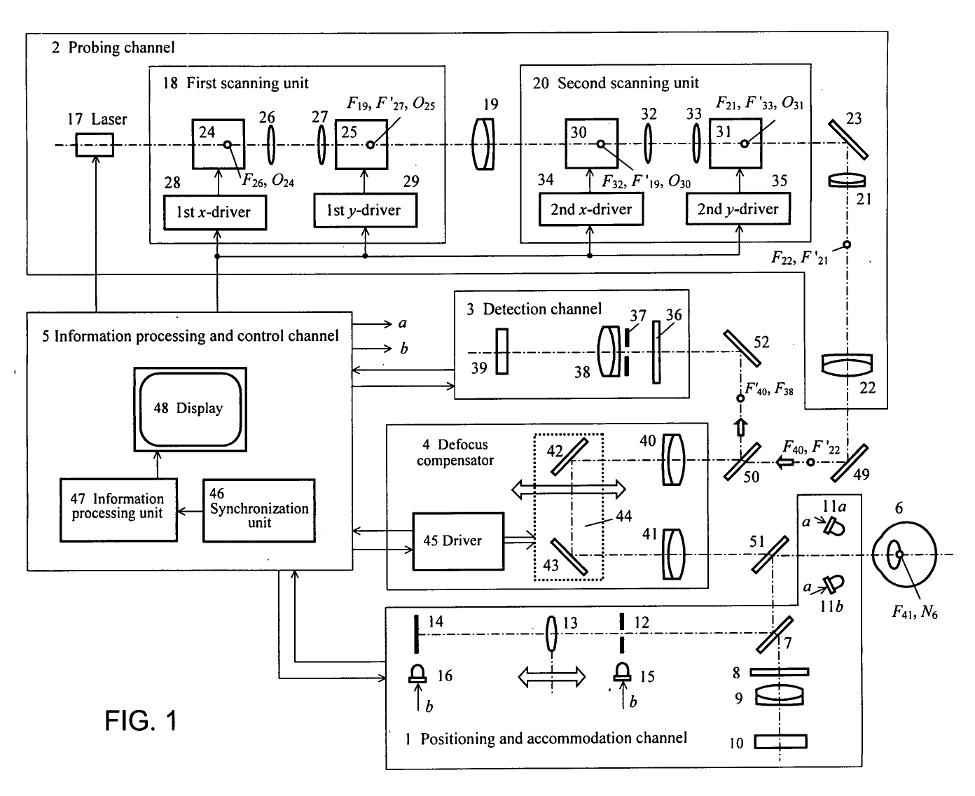

As shown in FIG. 1, the device contains a positioning and accommodation channel 1, a probing channel 2, a detection channel 3, a defocus compensator 4, and an information processing and control channel 5. The eye 6 is the object of investigation.

The positioning and accommodation channel 1 consists of a beam-splitter 7, a filter 8, an objective lens 9, an imaging camera 10 which can be a TV camera. Several sources of light 11, for example, light emitting diodes (LEDs) are installed in front of the eye 6. Two of them, 11a and 11b are shown in the FIG. 1. The positioning and accommodation channel 1 also includes a near target 12, a lens 13, and a far target 14. The lens 13 is movable along the optical axis. The near target 12 is illuminated by a source of light 15, and the far target—by a source of light 16. Said sources of light can also be LEDs.

The probing channel 2 consists of a laser 17, a first scanning unit 18, a collimating lens 19, a second scanning unit 20, a reflecting mirror 23, two lenses 21 and 22 composing a telescope. The first scanning unit 18 consists of a first x-deflecting acousto-optic crystal 24 and a first y-deflecting acousto-optic crystal 25. Between them, two lenses 26 and 27 are installed forming a telescope in such a way that the front focus F₂₆ of the lens 26 coincides with the center of scanning O₂₄ of the first x-deflecting acousto-optic crystal 24, and the back focus F′₂₇ of the lens 27 coincides with the center of scanning O₂₅ of the first y-deflecting acousto-optic crystal 25. A first x-driver 28 is electrically connected to the first x-deflecting acousto-optic crystal 24, and a first y-driver 29 is electrically connected to the first y-deflecting acousto-optic crystal 25.

The second scanning unit 20 consists of a second x-deflecting acousto-optic crystal 30 and a second y-deflecting acousto-optic crystal 31. Between them, two lenses 32 and 33 are installed forming a telescope in such a way that the front focus F₃₂ of the lens 32 coincides with the center of scanning O₃₀ of the second x-deflecting acousto-optic crystal 30, and the back focus F′₃₃ of the lens 33 coincides with the center of scanning O₃₁ of the second y-deflecting acousto-optic crystal 31. A seconf x-driver 34 is electrically connected to the second x-deflecting acousto-optic crystal 30, and a second y-driver 35 is electrically connected to the second y-deflecting acousto-optic crystal 31.

The collimating lens 19 is installed between the first scanning unit 18 and the second scanning unit 20 so that its front focus coincides with the center of scanning O₂₅ of the first y-deflecting acousto-optic crystal 25, and its back focus coincides with the center of scanning O₃₀ of the second x-deflecting acousto-optic crystal 30.

The lens 21 is installed with its front focus F₂₁ coinciding with the center of scanning O₃₁ of the second y-deflecting acousto-optic crystal 31. To meet the requirements of the telescope, its back focus F′₂₁ coincides with the front focus F₂₂ of the lens 22. The mirror 23 does not play any principal role but to bend the optical axis of the probing channel 2 for convenience of the construction.

The detection channel consists of the following sequentially installed components: a polarization filter 36, an aperture stop 37, an objective lens 38, and a position-sensing detector (PSD) 39. The position-sensing detector can be of any known type. The best solutions can be a two-dimensional structure, for example, of the CCD type, or two orthogonal linear multi-element detector arrays. In the last case, the detection channel is to be divided into two sub-channels, in which two cylindrical lenses form the projections for the orthogonal detector arrays.

The defocus compensator 4 consists of two lenses 40 and 41 forming a telescope. Two mirrors 42 and 43 form a mirror unit 44. The mirrors are oriented at 45 degrees to the optical axis so that they bend the optical axis 180 degrees to its initial direction. As a version, this unit can be made solid with two reflecting surfaces substituting the mirrors 42 and 43. The unit 44 is movable in the direction to or from the lenses 42 and 43 changing in this way the distance between the lenses 40 and 41. A driver 45 is electromechanically connected to the mirror unit 44.

The information processing and control channel 5 consists of a synchronization unit 46, an information processing unit 47, and a display 48. Inside the channel 5, the synchronization unit 46 is electrically connected to the information processing unit 47 and the display 48, as well as the output of the information processing unit 47 is electrically connected to the display 48. The information processing and control channel 5 has electrical connections to the laser 17, drivers 28, 29, 34, and 35 of the probing channel 2. Said channel 5 has two-way electrical connections with the positioning and accommodation channel 1, the detection channel 3, and the defocus compensator 4. Through the wires a, the channel 5 has electrical connections with the eye illuminating sources of light 11 (11a and 11b are shown in FIG. 1) and through the wire b—with the target illuminating source of light 14.

Outside the channels, there are mirrors, directing the ingoing and outgoing beams of light, and beam-splitters, optically interconnecting the channels. A totally reflecting mirror 49 bends the optical axis by 90 degrees to direct the laser beam from the probing channel 2 into the eye 6 through the beam-splitter 50, the defocus compensator 4, and another beam-splitter 51. The beam-splitter 50 has no difference in spectral transmission and reflection. The beam-splitter 51 has high transmission of laser radiation from the channel 2, and high reflection of light in the spectral regions of the imaging camera 10 and LEDs 15 and 16. The mirror 52 is a totally reflecting mirror. The mirrors 23, 49, and 52 play an auxiliary role to bend the optical axis, and they may not be present in the construction if there is no construction expediency.

On the way into the eye, the back focus F′₂₂ of the lens 22 coincides with front focus F₄₀ of the lens 40. On the way from the eye to the detection channel, the back focus F′₄₀ of the lens 40 coincides with the front focus F₃₈ of the lens 38. The eye 6 should be positioned in front of the lens 41 so that the back focus F′₄₁ of the lens 41 coincides with nodal point N₆ of the optical system of the eye.

Before the aberration measurement starts, the instrument and the eye should be correctly positioned. Firstly, the distance to the eye should correspond to the coincidence of the back focus F′₄₁ of the lens 41 with the nodal point N₆ of the optical system of the eye. This procedure is usually exercised indirectly being substituted by focusing of the image of the iris on the imaging camera 10. The eye is illuminated by a source or several sources of light, e.g., by the LEDs with the maximum of irradiation in the infrared. As such, AlGaAs LEDs can be used with the peak wavelength 910 nm. The distance of the focused image from the imaging camera 10 should correspond to the coincidence of the back focus F′₄₁ of the lens 41 with the nodal point N₆ of the optical system of the eye.

Secondly, the visual axis of the eye should be aligned with the optical axis of the instrument. To uniquely achieve this goal, the centers of the near target 12 and the far target 14 should be positioned on the optical axis of the instrument. Through said near target 12, the patient should see the far target 14, their overlaid centers should coincide. One of the possible embodiments of the near target 13 can be an opening in the non-transparent plate. Another embodiment of the near target 12 can be a tube, through which the far target 14 can be observed. During the process of alignment, the near target 12 is illuminated with the visible light of the LED source 15. It could be of any visible color or of a mixture of colors. In one of the embodiments, the far target 14 is illuminated by red light, and the near target 12—by green light. Any other combination of visible colors is possible from LEDs 15 and 16. Accommodation adjustment is provided by the movement of the lens 13 that can be also a more complicated component like a Badal optometer. Its construction is not principal from the point of view of the present invention. Any other design of the positioning and accommodation channel 1 can be implemented for the purposes of this invention.

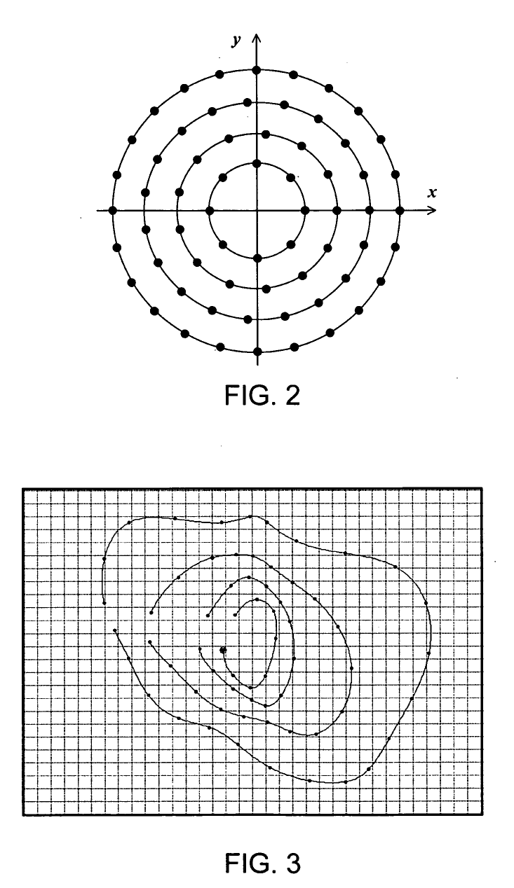

Aberration measurement of the properly positioned eye proceeds in two stages, the first of which is the preliminary stage, and the second is the main one. During the preliminary stage, the second scanning unit 20 is set to the zero-deflection position, i.e., the laser beams exiting in sequence from the collimating lens are projected in the eye in the same manner as in the regular ray tracing aberrometer described elsewhere, for example, in the U.S. Pat. No. 6,932,475. Each beam entering the eye is parallel to the optical axis of the instrument and to the visual axis of the eye. Beam crossings of the plane perpendicular to the optical axis of the aberrometer at the entrance of the eye are shown in FIG. 2. Typical number of beam positions is 64 to 256.

The laser 17 controlled from the information processing and control channel 5 emits a narrow beam of radiation directed to the input of the first scanning unit 18 which deflects the beam in x and y directions. The wavelength of the laser is not a specificity of this invention. Different considerations can be taken into account when choosing the wavelength. For example, invisible laser light (infrared) will make the procedure of aberration measurement patient-friendly. If the LEDs 11 emit at 910 nm, the wavelength of the laser 17 chosen in the range 780-810 nm will be quite appropriate.

There is no difference in which sequence the crystals 24 and 25 are installed. For distinctness, in the layout of FIG. 1, the laser beam enters primarily the first x-deflecting acousto-optic crystal 24. The angle of deflection in it is controlled by the information processing and control channel 5 through the first x-driver 28. Usually, the driver is a frequency synthesizer with the output stage driving the acousto-optic crystal. The crystal is configured to form a Bragg cell in which, due to diffraction on a regular structure excited by an acoustic wave, the deflection takes place of which a specific order (usually the first one) is selected. The angle of deflection is proportional to the synthesized frequency. For the material of the acousto-optic crystal, paratellurite (TeO₂) is a good candidate.

The duration of keeping the beam in a certain position is enough to have the order of milliseconds (i.e., 1-10 ms). Transition time of switching from one position to another is of the order of microseconds (typically, 1-10 μs). In the industrially manufactured ray tracing aberrometer (e.g., iTrace of the Tracey Technologies, Houston, Tex.), the total time of probing the whole aperture of the eye in 64-256 points is 100-250 ms (the number of entrance points and the exposure time are varied by the software).

A similar procedure is performed in y direction using the first y-deflecting acousto-optic crystal 25, controlled from the information processing and control channel 5 through the first y-driver 29. The design of the first y-deflecting acousto-optic crystal 25 is the same as that of the first x-deflecting acousto-optic crystal 24, except its 90-degree turn around the optical axis in regard to the first x-deflecting acousto-optic crystal 24. The structure and the functioning of the first y-driver 29 are the same as those of the first x-driver 28. The telescope consisting of the lenses 26 and 27 transposes the equivalent center of scanning O₂₄ in the crystal 24 into the equivalent center of scanning O₂₅ in the crystal 25. It is to be noted that both x and y control signals are applied to the crystals 24 and 25 simultaneously, thus deflecting the laser beam in a required direction having x and y components.

Since the equivalent center of scanning O₂₄ of the crystal 24 in x direction transposed into the center of scanning O₂₅ of the crystal 25 in y direction which is positioned in the front focus of the collimating lens 19, all beams exiting from the O₂₅ will have their axes parallel to the optical axis of the instrument after the lens 19. If the second scanning unit 20 is in the zero deflection mode, it will be equivalent to the plano-parallel plate thus keeping the axes of all beams parallel. The telescopes 21-22 and 40-41 (if the latter is in the a focal position) also keep the axes of all beams parallel. Under these conditions, as mentioned earlier with reference to FIG. 2, all beams enter the eye with their axes parallel to the optical axis of the instrument and parallel to the visual axis of the eye.

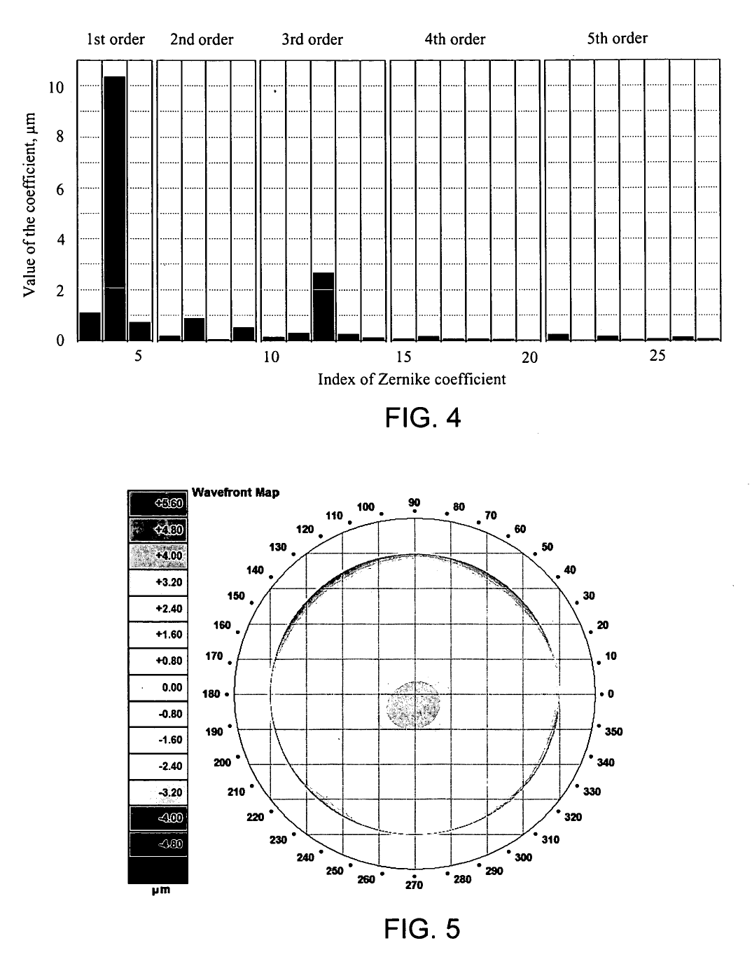

Any beam, entering the eye in a given moment of time, after hitting the retina will be scattered in it, this scattered light having a portion of light scattered in a backward direction. The back-scattered light will reach the detection channel 3 after passing the telescope 41-40 which in its confocal position relays the beam coming from the eye to the detection channel 3. Polarizing filter 36 selects only the component of light, whose polarization is orthogonal to the initial polarization of the light entering the eye. The aperture stop 37 restricts off-axis radiation. Objective lens 38 projects the radiation on the position-sensing detector 39 whose receptive surface is conjugated with the retina. In this way, the position of each laser spot on the retina can be measured and transferred to the information processing and control unit 5. A set of these spots constitutes a retina spot diagram of the type shown in FIG. 3. Each entrance point finds its correspondence in the retina spot diagram. From these data, the parameters of refraction are calculated in the information processing unit 47. To get the distribution of these parameters over the entrance pupil of the eye, several approaches can be implemented like spline interpolation or approximation using polynomial expansions. The least squares technique is normally applied to get the approximation with Zernike polynomial coefficients. An example of five-order Zernike expansion calculated from the retina spot diagram is shown in FIG. 4. In this example, prevailing are the first order aberrations (defocus and astigmatism). FIG. 5 is an example of the wave front map reconstructed using the least squares technique of approximation.

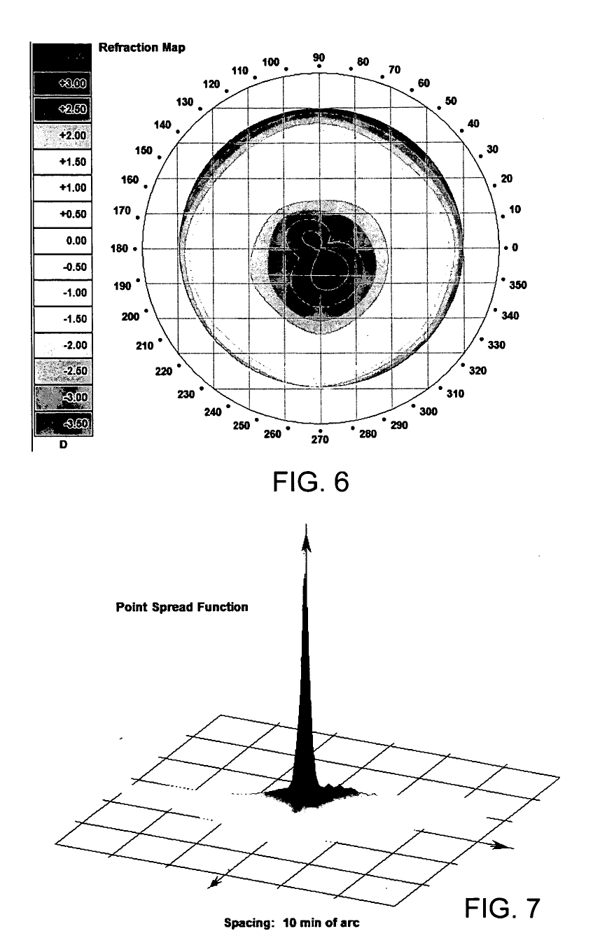

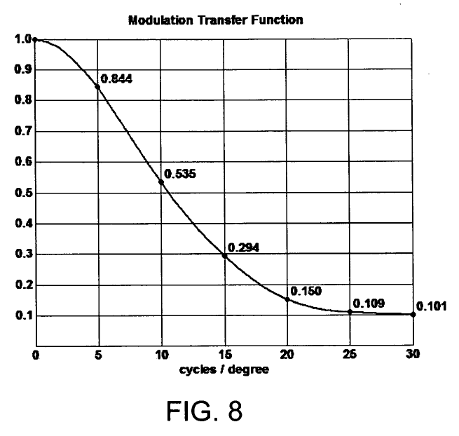

Any other derivative parameter can be calculated from the results of measurement. FIG. 6 demonstrates an example of the aberration map of the same patient. Calculated also are point spread function (FIG. 7) and modulation transfer function (FIG. 8). All these data are calculated and processed by the information processing unit 47 and are displayed by choice on the display 48.

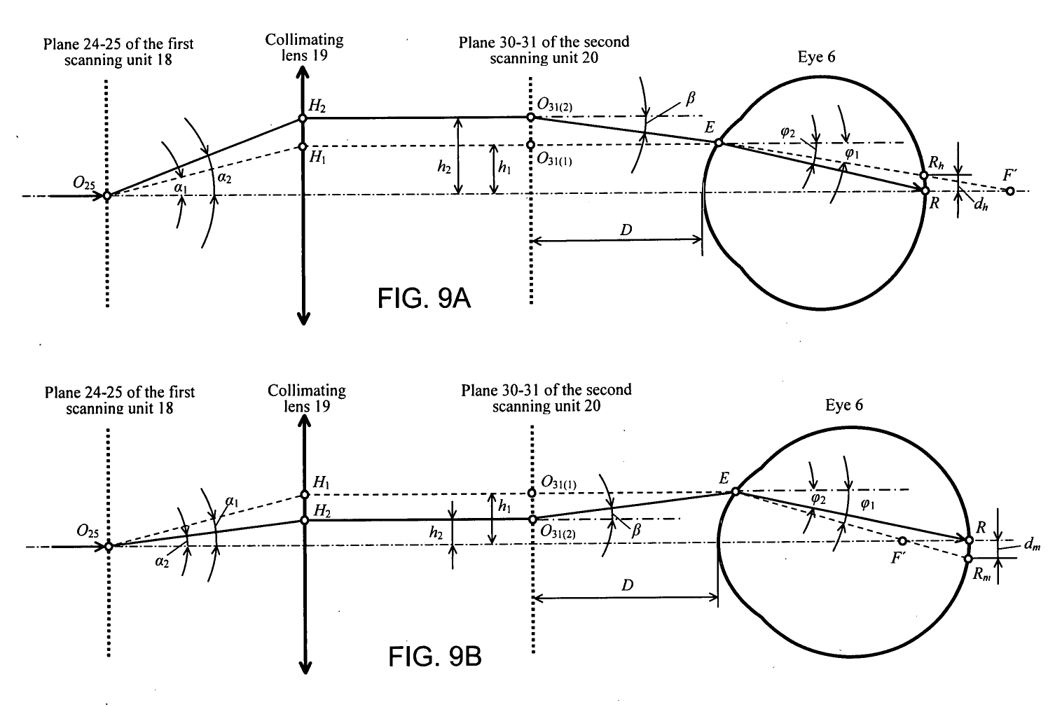

The parameters calculated and displayed as a result of the first, preliminary stage of measurement are correct only as a first approximation. This is because the light propagating in the eye in the back direction is influenced by the aberrations that distort (in the plane of position sensing detector 39) the positions of the laser spots on retina. There are two ways to avoid such distortions: (1) to exclude the distorting effects in eye media or, (2) to correct the tilt of the laser beam at its entrance into the eye making it to hit the retina in the point corresponding to the eye with no aberrations thus compensating the refractive error for each entrance point. The first approach requires expensive active optics initially used in astronomy and precise laser radar systems and weapons. FIG. 9 explains the second technique implemented in the schematic layout of FIG. 1.

Planes 24-25 and 30-31 perpendicular to the optical axis in which the beams change their directions in the acousto-optic crystals are shown as dotted lines. The centers of scanning are denoted as O₂₅ and O₃₁ correspondingly, taking into account that O₂₄ can be regarded as coinciding with O₂₅, and O₃₀—with O₃₁. The collimating lens 19 is shown as a thin lens. In the preliminary stage of measurement, the laser beam exiting from the point O₂₅ at an angle al crosses the collimating lens 19 in the point H₁ and follows further in parallel to the optical axis at the height h₁. In the first stage of measurement, the second scanning unit 20 is in the zero deflecting position. It means that after crossing the plane 30-31 in the point O₃₁₍₁₎ the beam continues to follow in parallel to the optical axis and reaches the eye in the point E, after which the beam will be bent at an angle φ₁. In the case of a hyperopic eye with the focal point F′ behind the retina (FIG. 9A), the retina will be hit in the point Rₕ at the distance dₕ off the optical axis. In the case of a myopic eye with the focal point F′ in front of the retina (FIG. 9B), the latter will be hit in the point Rₘ at the distance dₘ off the optical axis.

The distances dₕ or dₘ off the optical axis are measured with the position sensing detector 39. The results of measurements include the errors due to distortions in the back direction. Therefore, the results of calculations can be regarded as the first approximation, which can be used as initial data for compensation of the aberrations measured in a given point E of entrance into the eye. Compensation of aberrations means that the beam entering the eye in the point E should be bent at an angle φ₂ instead of φ₁ to hit the retina in the point R corresponding to the crossing of the retina by the visual axis (instead of Rₕ or Rₘ). For the sake of simplification, we do not discuss here the peculiarities of the optical system of the eye, simply suggesting that the visual axis crosses the retina in the point referred to as the central point of macula. To be bent at the angle φ₂, the beam should reach the point E at an angle β to the optical axis. To meet this condition, the beam when crossing the plane 30-31 must exit from the point O₃₁₍₂₎ at the height h₂ from the optical axis. Continuing this logic, the beam should cross the collimating lens 19 at the same height h₂ in the point H₂ thus having the initial angle α₂ of deflection when exiting from the point O₂₅ of the plane 24-25 (instead of α₁). The described beam transforms in a single plane of drawing are only an example, all these transforms usually take place in the 3D space.

The second, main stage of measurements proceeds as follows. For each entrance point E, angles α₂ and β are calculated, and laser beam is directed into the eye in point by point manner. First, the beam tilt β is calculated, then, using the back-tracing, its coordinates at the exit of the second scanning unit 20 are calculated. In the simplified drawing of FIG. 9, it corresponds to the point O₃₁₍₂₎. In this simplification, the height h₂ is the same at the entrance and at the exit of the second scanning unit 20. In reality, the thickness of the crystals should be taken into account, and the entrance coordinates in the second scanning unit should be calculated. With the knowledge of height h₂, one may come to the calculations of the angle α₂ at which the beam should start from the point O₂₅ of the first scanning unit 18.

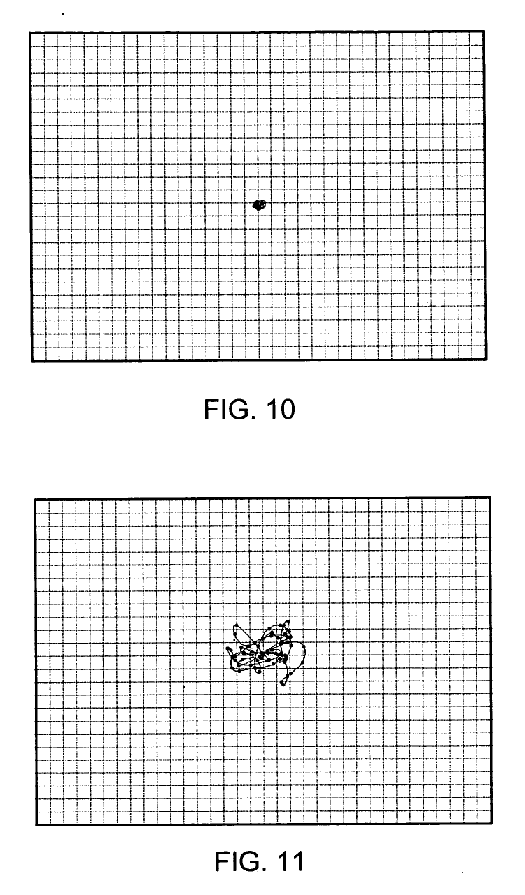

The procedures of detection and determining the position of each laser spot on retina, as well as wave front calculations are the same as in the first stage of measurements with the only difference that the new tilts of the laser beam at the eye entrance points should be taken into account (that could be non-zero as referred to the optical axis). As a result of this main stage, all the spots on retina should be concentrated in the point R, if there were no error in determination of the positions Rₕ or Rₘ. An example of the retina spot diagram acquired in the second stage for an eye with moderate aberrations is demonstrated in FIG. 10. In a highly aberrated eye, the spots on retina will be dispersed around the point R, still in the shorter distances as compared to the preliminary stage. An example of such retina spot diagram reconstructed at this stage is shown in FIG. 11. Said diagram corresponds to the errors of measurements that were not compensated during the second stage. They may originate from the distortions on the way of the light back from the eye. To compensate for these errors, the next iteration should be applied.

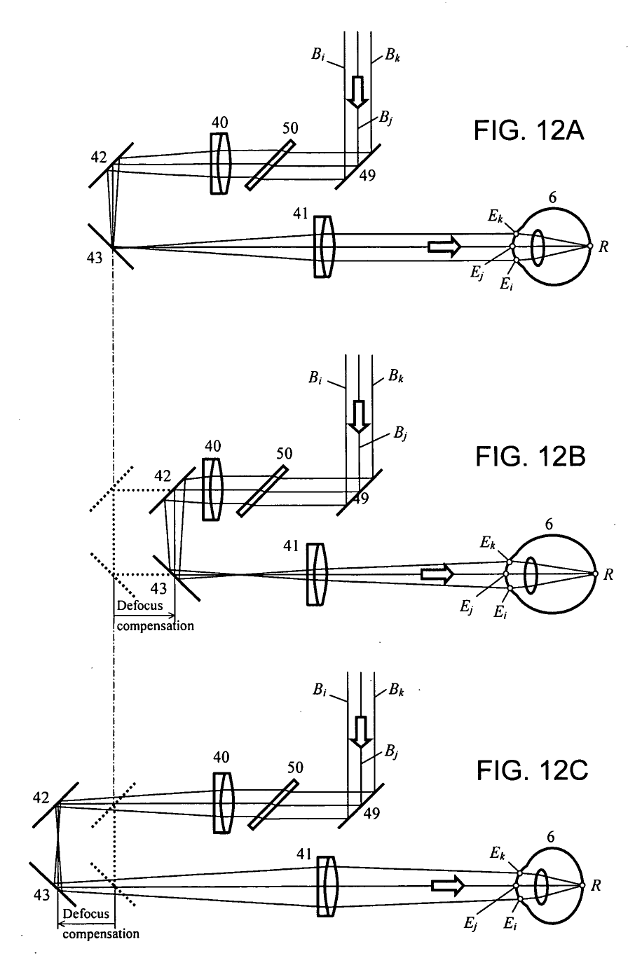

Still another procedure can be implemented with the device of FIG. 1. To lessen the dispersion of spot distances from the axis in the second stage of measurements, defocus can be compensated using the adjustable telescope 40-41. This is done changing the distance between the lenses 40 and 41 with the movable platform 44 containing the mirrors 42 and 43. Positions of the mirrors 42 and 43 for different cases are presented in FIG. 12, where FIG. 12A corresponds to an emmetropic eye, FIG. 12B—to a myopic eye, FIG. 12C—to a hyperopic eye. For the sake of simplification, only defocus is shown in these drawings without any higher order aberrations. From the whole set of probing beams, three are shown in the drawing: Bᵢ, Bⱼ, and Bₖ. Their points of entrance are correspondingly: Eᵢ, Eⱼ, and Eₖ. Note that in all three cases, these points are the same for all mentioned beams Bᵢ, Bⱼ, and Bₖ. The changed are only the tilts of the beams at the entrance into the eye making them to reach the retina in the same point R.

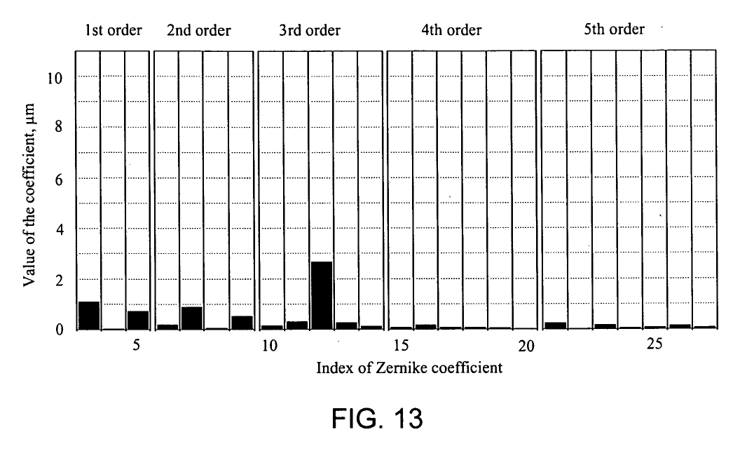

If the defocus is compensated completely, coefficient Z₄ in the Zernike decomposition will be equal to zero as demonstrated in FIG. 13. The movable platform 44 (FIG. 1) is shifted by the driver 45. Said driver is controlled by the signals from the information processing and control channel 5. To work out the control signals, the data from the preliminary stage are used. The signal may be proportional to the Z₄ component of the Zernike decomposition, or it can be determined in a simpler way from several points of entering into the eye. Normally, four points may be enough. It means, that there is no necessity to go through all the cycle of measurements in all entrance points, and the procedure can be designed in such a way, that only four points are probed first to deliver the data to the information processing and control channel 5 for working out the amount of shift for the platform 44. If the component Z₄ is not compensated completely in the second stage, the results of the second stage of measurements will contain this non-compensated portion of Z₄.

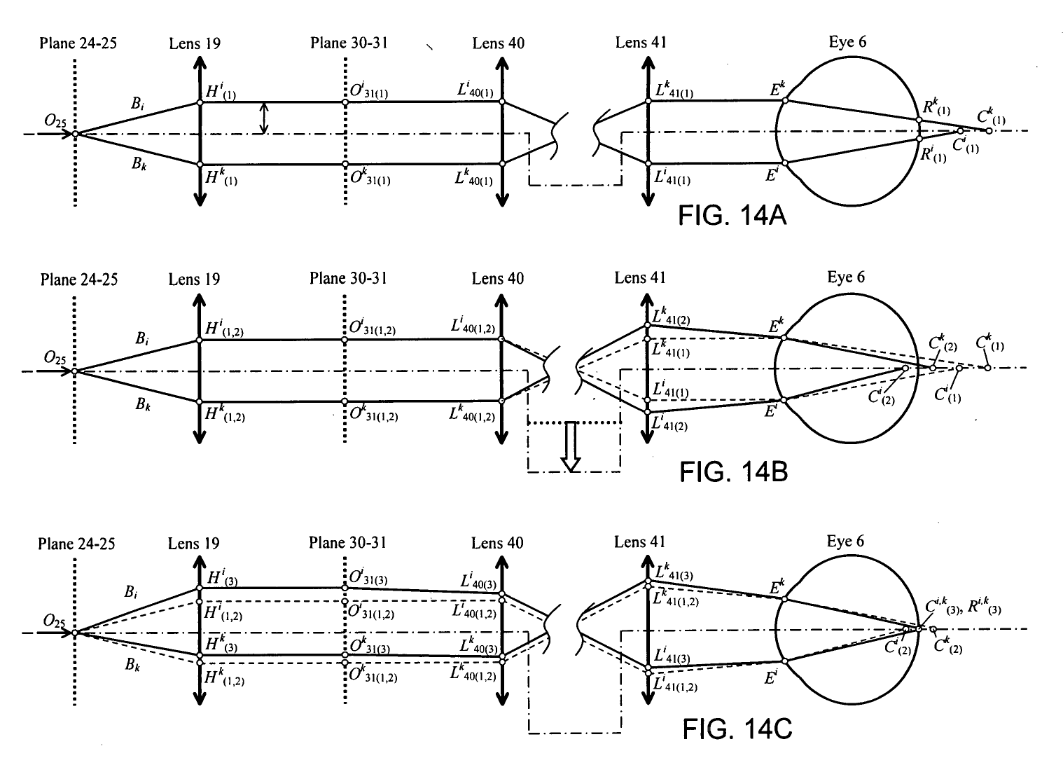

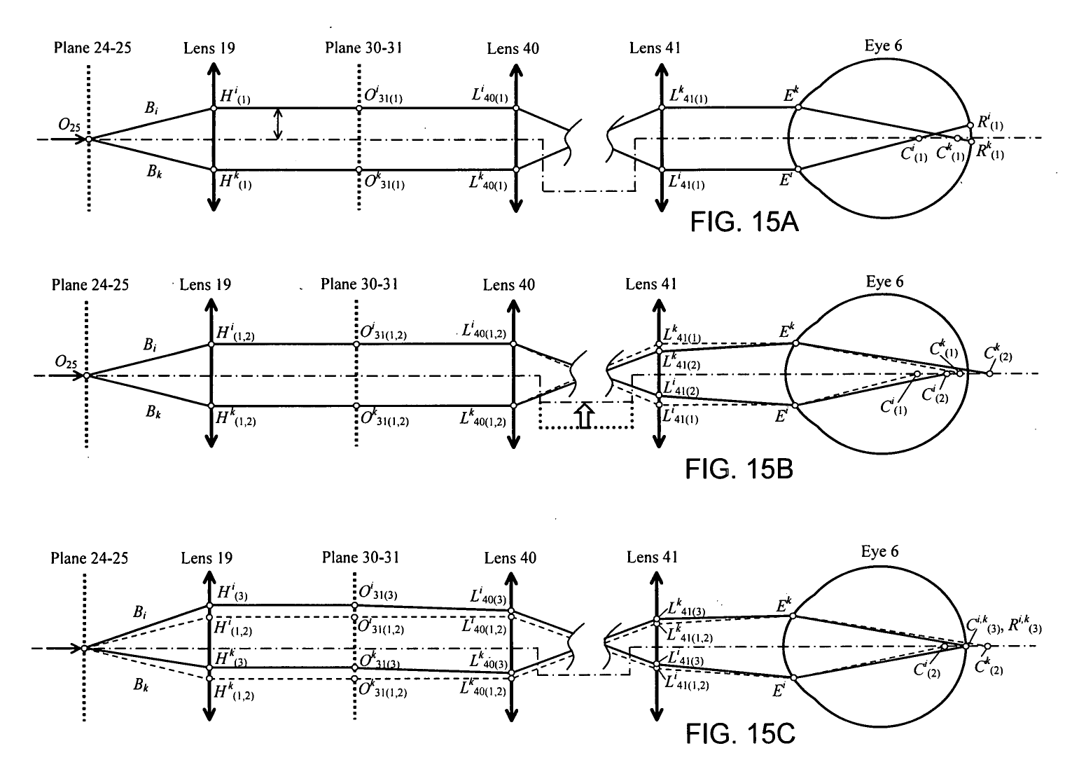

FIGS. 14 and 15 show the entire chain of beam transformations including scanning units 18 and 20 and the defocus compensator 4. FIG. 14 corresponds to a hyperopic eye, FIG. 15—to a myopic eye. Two beams are analyzed: Bᵢ and Bₖ. The points in the drawings are labeled by the letters with superscripts (i and k) corresponding to the beams Bᵢ and Bₖ and subscripts corresponding to the characteristic planes (if without brackets) and to the stage of transformation (in brackets): (1) is for the preliminary stage (zero deflection in the second scanning unit and no defocus compensation in the defocus compensator 4); (2) is for defocus compensation in the defocus compensator 4; (3) is for the main stage with defocus compensation by the defocus compensator 4 and compensation of higher order aberrations using both scanning units 18 and 20.

Dotted lines in FIGS. 14B and 15B denote traces of the beams Bᵢ and Bₖ as they were in the preliminary stage shown in FIGS. 14A and 15A correspondingly. Similarly, dotted lines in FIGS. 14C and 15C denote traces of the beams Bᵢ and Bₖ as they were with only defocus compensation shown in FIGS. 14B and 15B correspondingly.

Movable mirrors 42 and 43 are not shown. The shifts of these mirrors are shown in the drawings as changed lengths of the bent chain lines between the lenses 40 and 41. For all that, the arrows in the space between lenses 40 and 41 in FIGS. 14B and 15B denote direction of the shifts of the movable mirrors 42 and 43.

Let us track the beams for different stages. In the hyperopic eye in the preliminary stage (FIG. 14A), the trace of the beam Bᵢ is O₂₅-Hⁱ₍₁₎-Oⁱ₃₁₍₁₎-Lⁱ₄₀₍₁₎-Lⁱ₄₁₍₁₎-Eⁱ-Rⁱ₍₁₎, and if keeping on, it would cross the optical axis in the point Cⁱ₍₁₎. The beam Bₖ follows the trace O₂₅-H^{k}₍₁₎-O^{k}₃₁₍₁₎-L^{k}₄₀₍₁₎-L^{k}₄₁₍₁₎-E^{k}-R^{k}₍₁₎, and if keeping on, it would cross the optical axis in the point C^{k}₍₁₎. In the myopic eye in the preliminary stage (FIG. 15A), the trace of the beam Bᵢ is O₂₅-Hⁱ₍₁₎-Oⁱ₃₁₍₁₎-Lⁱ₄₀₍₁₎-Lⁱ₄₁₍₁₎-Eⁱ-Cⁱ₍₁₎-Rⁱ₍₁₎, crossing the optical axis in the point Cⁱ₍₁₎ before it hits the retina in the point Rⁱ₍₁₎. The beam Bₖ follows the trace O₂₅-H^{h}₍₁₎-O^{k}₃₁₍₁₎-L^{k}₄₀₍₁₎-L^{k}₄₁₍₁₎-E^{k}-C^{k}₍₁₎-R^{k}₍₁₎.

With involvement into functioning of the defocus compensator 4, in the case of hyperopic eye (FIG. 14B), the distance between the lenses 40 and 41 grows, and the traces of the beams Bᵢ and Bₖ cross the optical axis in the points Cⁱ₍₂₎ and C^{k}₍₂₎ shifted to the front of the eye as compared to the positions of the points Cⁱ₍₁₎ and C^{k}₍₁₎. In the case of myopic eye (FIG. 15B), the distance between the lenses 40 and 41 is made shorter, and the beams Bᵢ and Bₖ cross the optical axis in the points Cⁱ₍₂₎ and C^{k}₍₂₎ shifted to the back of the eye as compared to the positions of the points Cⁱ₍₁₎ and C^{k}₍₁₎. Note, that defocus compensator shifts crossing points C all together, “collectively”.

Switching on the second scanning unit 20, “personalizes” these shifts for each beam. In the examples of FIGS. 14C and 15C, the point Cⁱ₍₂₎ is shifted to the position Cⁱ₍₃₎ (in the direction to the back of the eye), and the point C^{k}₍₂₎ is shifted to the position C^{k}₍₃₎ (in the direction to the front of the eye), both positions coinciding with each other (being labeled as C^{i,k}₍₃₎) and with the positions of the points Rⁱ₍₃₎ and R^{k}₍₃₎, being labeled as R^{i,k}₍₃₎. It is to be mentioned that when switching on the second scanning unit 20, the traces of the beams before they enter said scanning unit 20 should be recalculated. It means that angles of deflection in the first scanning unit 18 should be corrected as well. The new traces of the beams Bᵢ and Bₖ will be O₂₅-Hⁱ₍₃₎-Oⁱ₃₁₍₃₎-Lⁱ₄₀₍₃₎-Lⁱ₄₁₍₃₎-Eⁱ-{C^{i,k}₍₃₎, R^{i,k}₍₃₎} and O₂₅-H^{k}₍₃₎-O^{k}₃₁₍₃₎-L^{k}₄₀₍₃₎-L^{k}_{41( 3)}-E^{k}-{C^{i,k}₍₃₎, R^{i,k}₍₃₎} correspondingly,

If the aberrations are so big that they distort results of measurements of laser spots positions on retina and therefore, the aberrations are not compensated completely in the main stage of measurements, or the value of defocus compensation is not correct enough, an additional iterative step of measurement may be necessary. In this additional step, only the “individual” correction of beam directions should be implemented.

With the proposed principle, it is easier to follow the dynamics of eye aberrations, because in the process of such measurements, only small changes are to be measured. It can be done more accurately in comparison with the measurement of small changes of large background values.

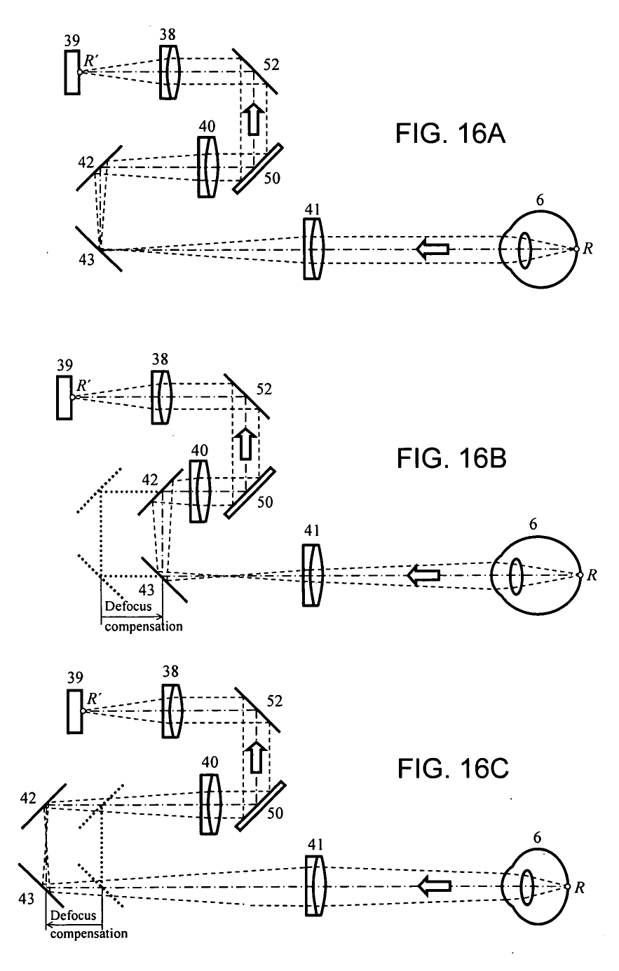

Defocus compensation tightening the scatter of laser spots on retina is important also for focusing the images of said laser spots on the position sensitive detector 39. It makes the procedure of measuring spot coordinates more accurate. FIG. 16 shows how the radiation exiting from the eye is focused on the position sensitive detector 39 for different eyes. FIG. 16A corresponds to the emmetropic eye, FIG. 16B—to the myopic eye, FIG. 16C—to the hyperopic eye. Dotted lines in FIGS. 16B and 16C show initial positions of the mirrors 42 and 43 determined for the emmetropic eye.

The laser beam projected into the emmetropic eye, scattered on the retina and re-radiated in the back direction, exits the eye as a parallel beam with the diameter corresponding to the size of the pupil. Objective lens 38 is designed to focus a parallel beam in the plane of a photosensitive surface of the PSD 39.

If the eye is myopic, the exiting beam is converging (FIG. 16B). To compensate for this convergence and to make the beam parallel at the entrance of the objective lens 38, the distance between the lenses 40 and 41 is made shorter. And it is just the same as when compensating the defocus at beam projecting.

When the eye is hyperopic, the exiting beam is diverging (FIG. 16C). To compensate for this divergence and to make the beam parallel at the entrance of the objective lens 38, the distance between the lenses 40 and 41 is made longer, the same as when compensating the defocus at beam projecting.

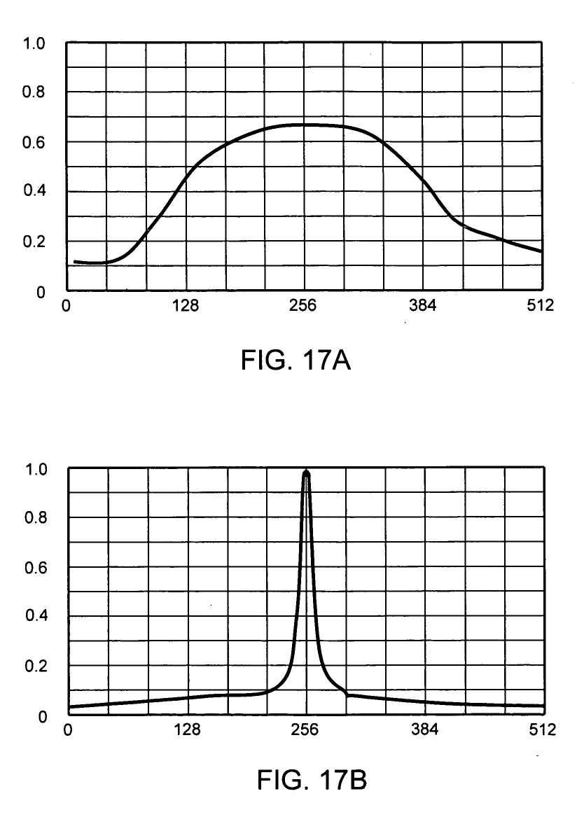

Diagrams of FIG. 17 demonstrate the shape of intensity distribution in the plane of the PSD 39. As an example, not restricting the field of this invention, horizontal axis is labeled with the numbers of elementary detectors of the 512-element linear array. Shown is the diagram from one of two such arrays oriented orthogonally to each other. As mentioned earlier, a two-dimensional detecting matrix (like CCD) can also be used instead of two linear arrays. Vertical axis is labeled in magnitudes of the signal from each element (normalized).

FIG. 17A corresponds to the signal from the eye of the patient with 10 diopter non-compensated hyperopia. FIG. 17B shows how steeper becomes the signal, when ametropia is compensated with the defocus compensator 4.

What is claimed is:

1. Device for wave front conjugated ray tracing aberrometry, containing a positioning and accommodation channel, a probing channel, a detection channel, and an information processing and control channel,
said positioning and accommodation channel consisting of a beam-splitter, a filter, an objective lens, an imaging camera, eye illuminating light sources installed in front of the eye, a near target, a lens movable along the optical axis, and a far target,
said probing channel consisting of a laser, a first scanning unit, a collimating lens, and a telescope, said first scanning unit having sequentially installed a first x-deflecting acousto-optic crystal connected to a first x-driver, a telescope, and a first y-deflecting acousto-optic crystal connected to a first y-driver,
said detection channel consisting of sequentially installed a polarization filter, an aperture stop, an objective lens, and a position-sensing detector,
said information processing and control channel consisting of a synchronization unit, an information processing unit, and a display, said synchronization unit being electrically connected to said information processing unit and said display, the output of said information processing unit being electrically connected to said display, said information processing and control channel having electrical connections to said probing channel, said positioning and accommodation channel, and said detection channel,
said positioning and accommodation channel, said probing channel, and said detection channel having common optical axis, and being optically connected through beam splitters,
wherein between the collimating lens and the telescope of said probing channel, a second scanning unit is installed consisting of a second x-deflecting acousto-optic crystal connected to a second x-driver, a telescope, and a second y-deflecting acousto-optic crystal connected to a second y-driver, both said second x-driver and said second y-driver are connected to said information processing and control channel.

2. Method for wave front conjugated ray tracing aberrometry based on consecutive projections of thin laser beams on retina through a set of points of the eye entrance aperture, measurement of the coordinates of the projected laser spots on retina, calculation of the wave front tilt in each entrance point from the known coordinates of the entrance points and measured coordinates of the projected laser spots on retina, reconstruction of the wave front map using mathematical methods of interpolation or approximation and calculation of other derivative characteristics comprising the conjugation of the laser beam tilt at the entrance into the eye to compensate for the tilt induced by the aberrations along the beam path in the eye in the steps of:
a) calculation of the beam tilt at the entrance into the eye in a point with known coordinates;
b) back-tracing the beam to determine its coordinates at the exit of the second scanning unit;
c) calculation of the entrance coordinates in the second scanning unit;
d) calculation of the angle of deflection in the first scanning unit;
e) applying the voltages to the crystals of the first scanning unit with the frequencies corresponding to the angles of deflection calculated in the step (d);
f) applying the voltages to the crystals of the second scanning unit with the frequencies corresponding to the angles of deflection calculated in the step (a);
g) repeating the steps of consecutively projecting thin laser beams on retina through the initially designated set of points of eye entrance aperture at the angles defined in steps (a) and (d), measuring the coordinates of the projected laser spots on retina, calculating the wave front tilt in each entrance point from the known coordinates of the entrance points, known laser beam tilts at the entrance into these points, and measured coordinates of the projected laser spots on retina, reconstructing the wave front map and other derivative characteristics using mathematical methods of interpolation or approximation.

3. A method as claimed in claim 2, wherein the steps (a)-(g) are repeated iteratively until the deviation of the laser spots on retina from a central position is less than specified in advance.

4. A device as claimed in claim 1, wherein a defocus compensator consisting of two lenses forming a telescope and two reflecting surfaces between said two lenses is installed at the entrance of the eye on the path common for the probing channel and for the detecting channel, said reflecting surfaces being oriented at 45 degrees to the optical axis, the back focus of the last lens of said defocus compensator coincides with the nodal point of the eye.

5. A method as claimed in claim 2, wherein the conjugation of the laser beam tilt at the entrance into the eye compensating for the tilt induced by the aberrations along the beam path in the eye is performed separately for defocus component of the eye aberrations—by the defocus compensator and for all the rest of aberration components—by the first and the second scanning units.

6. A device as claimed in claim 1, wherein a magnifying telescope is installed at the exit of the second scanning unit, the front focus of its first lens coinciding with the center of scanning of the second scanning unit.
